(19) United States
(12) Patent Application Publication
(10) Pub. No.: US 2025/0174103 A1
(43) Pub. Date: May 29, 2025

(54) SYSTEMS AND METHOD FOR CONTROLLING NOTIFICATIONS DEVICE VIA SURFACE VIBRATIONS

(71) Applicant: BLUE OAK COMPANY LIMITED, Bangkok (TH)

(72) Inventors: KUNANON KITTIPUTE, Bangkok (TH); PATTANA LEE, Bangkok (TH); SOMBOON CHIEWCHARNPIPAT, Bangkok (TH)

(21) Appl. No.: 18/841,426

(22) PCT Filed: Feb. 27, 2023
(86) PCT No.: PCT/TH2023/000002

§ 371 Date: Aug. 26, 2024

(30) Foreign Application Priority Data
Feb. 26, 2022 (TH) 2201001221

(51) Int. Cl. G08B 21/04

(52) U.S. Cl. CPC G08B 21/043; G08B 21/0423; G08B 21/0461

(57) ABSTRACT

This invention is the development of the system and method for controlling notifications device via surface vibrations, the system for controlling the notifications device via surface vibrations caused by the subject comprises a geophone sensor or a sensor for detecting vibration from the surface placed in contact with the surface, a processing unit which the vibration from the sensor is brought into the processing unit to improve it as the first vibration pattern and compared to determined vibration patterns, then send commands to control notifications device and the notifications device with a receiving module to receive commands from processing unit. The system and method for controlling notifications device via surface vibrations will effectively help in emergency notifications even if the user is unable to move or lose consciousness, resulting in timely assistance and deactivation of false alarms as well.

### FIELD OF THE DISCLOSURE

The present invention relates to a sensor engineering and technology related to system and method for controlling notifications device via surface vibrations.

### BACKGROUND OF THE DISCLOSURE

Aging Society is on the rise in both developing and developed countries including Singapore, Japan, South Korea, and Thailand, etc., while the birth rate and the number of the working-age population continue to drop; what follows for the elderly population is health problems and chronic diseases. In addition, it was also found that in urban societies where their children still have to work outside, it is necessary to take care of the elderly to have a good quality of life, reduce the chances of accidents, and delayed treatment.

The application of various types of electronic devices or the installation inside the residence is, therefore, a good solution for such problems; however, the safety, convenience, proper and uncomplicated use factors, including privacy, are also factors to be considered. In addition, electrical or electronic equipment used inside the building is often equipped with a control panel at the body of device, or mounted in a limited number of points, which may be wired or wireless depending on how to signal between the control panel and device, which this control process is suitable for users in a condition that can access and control the control panel which is convenient and fast enough for the purpose and operation of that device. However, there are still many cases where the control panel at different points may not be able to be sufficiently accessible, for example, in an emergency case or user is not in a condition that can access the said control panel, for example, in a state of being unable to help oneself and requiring assistance, etc.

Currently, in the case of a call for assistance device, a solution may be to use a wall-mounted buzzer to request help. However, this method still has several drawbacks. For instance, the user may not be in the area with the buzzer when they need to press it. For example, if someone needs urgent help while choking in the middle of the room, they may be unable to walk and press the buzzer or stand up and reach for it, etc.

When using wearable devices to detect abnormalities in the elderly and signal to report these abnormalities to family members and/or medical personnel, several problems arise. These include the cooperation of the elderly, somewhat complicated usage, compatibility for sensitive skin, familiarity with the technology, and charging the devices for users.

The voice command technology can be used for the elderly to call for assistance. However, the system may make users feel violated in terms of privacy issues as if they were being eavesdropped on. Additionally, the system may become confused with commands for requesting help, leading to incorrect operations. There is an issue of accent involved, and the usage time may disturb other residents, for example, in case of signalling while another resident in the room is asleep or even the user may not be in a state where the voice can be clear or loud enough.

Video cameras are widely used to detect or transmit images that allow children to know about the activities of the elderly. However, privacy concerns still exist, as well as limitations to the installation location, such as in bathrooms or dark areas.

Therefore, efforts have been made to develop system and methods for effectively controlling notifications device without privacy issues, light restrictions, with high precision, reduced false alarms, and the ability to alert even if the user is unable to move or is unconscious. These systems must be convenient for users to allow the elderly to have a better quality of life and live alone more safely, which can reduce the likelihood of death or disability.

### SUMMARY OF THE DISCLOSURE

The system for controlling the notifications device via surface vibrations caused by the subject, comprising a sensor for detecting the vibration from the surface placed in contact with the surface, a processing unit which the vibration from the sensor is brought into the processing unit to improve it as the first vibration pattern and compared to determined vibration patterns, and then sends commands to control notifications device. The notifications device includes a receiving module to receive commands from the processing unit.

The method for controlling notifications device via surface vibrations generated by the subject comprising receiving the first vibration signal from the surface obtained from the sensor for detecting vibrations, processing the first vibration signal to the first vibration pattern, comparing the first vibration pattern to determined vibration patterns, and controlling notifications device based on the compatibility of the first vibration pattern and the determined vibration patterns.

The aim of this invention is to provide system and methods for controlling notifications device via surface vibrations that can effectively alert emergency services without privacy issues, light restrictions, and with fewer mistakes, even when the user is unable to move or unconscious. False alarms can also be effectively deactivated.

### BRIEF DESCRIPTION OF THE DRAWINGS

FIG. 1 is a diagram showing a pattern of workflow of systems for controlling notifications device via surface vibrations according to this invention.

FIG. 2 is a graph showing a pattern of vibration patterns caused by usual walking back and forth of the subject versus falling.

FIG. 3 is a graph showing vibration pattern caused by usual and unusual paces of the subject: (a) usual walking; (b) unusual walking with right foot pressure; (c) unusual walking with left foot pressure; and (d) abnormally slow walking.

FIG. 4 is a diagram showing a working radius of vibration detection of this invention.

### DETAILED DESCRIPTION OF THE EXEMPLARY EMBODIMENTS

### In this Description

The term “notifications device” or “notifying device” refers to any device, including electrical or electronic equipment, that operates by transmitting signals in the form of sound, light, text, image, vibration, or other similar or interchangeable forms. These devices can activate functions to inform any person or specified, designated individual of an unusual event, emergency, and/or need for assistance. Examples of such devices include anti-theft notifications device s, smoke/fire detectors, buzzers/rings, tablets, telephones, messaging devices, and more.

The term “vibration” refers to the motion or movement of the floor or surface back and forth relative to a reference point due to an external force applied to the floor or surface. In particular, it refers to the force caused by the subject or user that causes the floor or surface to vibrate with the same or different frequency, amplitude, count, and interval. For example, this can include the vibration caused by the movement of the room floor due to kinetic energy from falling down or the impact of a hand or foot, or any other part of the subject or user's body.

The term “compatibility of vibration pattern” refers to the degree of similarity between two sets of vibration signals in terms of their properties, such as frequency, amplitude, count, and interval. This degree of similarity can be adjusted according to the user's preferences to balance between accuracy and coverage.

This invention involves system and method for controlling notifications device via surface vibrations, in particular, vibration may be caused by paces, hitting with hands or foot on the surface where the device is placed. The vibrations from the floor are detected by sensor and further processed to know the characteristics of unusual paces or walking, falling down, accidents or emergency, and further alert.

### [Systems for Controlling Notifications Device Via Surface Vibrations]

The systems for controlling notifications device via surface vibrations, the system comprises:
A. Sensor: for detecting vibrations from the surface in contact with it.
B. Processing unit: the vibrations detected by the sensor are processed by the processing unit to create the first vibration pattern, which is then compared to the determined vibration patterns. The processing unit then sends commands to control the notifications device.
C. Notifications device with a receiving module to receive commands from the processing unit.

Wherein each component has additional details as follows.

[Sensor for Detecting Vibrations from Surface]

The sensor that is used is, preferably, a sensor that can detect vibrations from the surfaces, especially, the vibration caused by walking and/or falling down with frequency in a range of 1-200 Hz, for example, geophone sensor, which is suitable for detecting ground vibrations (Hons, M. S. (2008)). Seismic sensing: comparison of geophones and accelerometers using laboratory and field data (Unpublished master's thesis). University of Calgary, Calgary, AB. doi: 10.11575/PRISM/2190) by setting the vibration or vibration signal to vary according to the weight and shape of the subject, with the scope for specifically detecting vibrations or vibration detection radius, in a range of 3-5 meters, the vibration detection radius may be increased or adjusted by the properties of sensor applied (for example low/high gain sensitivity) and conductivity of surface material (for example, wooden floor and concrete floor have different conductivity). In addition, optionally, if the required room or area is larger than the detection radius, the devices may be increased to cover.

The parameters used to compare vibration patterns comprises amplitude, frequency, count, and interval, it can be seen that the vibration data obtained not only depends on the weight of the subject, but is also set to depend on other parameters to achieve accuracy, be able to distinguish between falling people and falling object on the ground, even if the falling object is heavy and/or large.

### [Vibration Data Processing Unit]

The processing unit comprises an amplifier that amplifies the vibration signals received from the sensor, an analog-to-digital converter that converts the analog vibration signals to digital signals, a microcontroller that analyzes and processes the digital signals, a network module that enables communication through various networks such as WiFi and Bluetooth, and a power supply module that supplies electricity. Additionally, it may comprise a storage unit or additional memory for data storage (if any).

The amplification device amplifies the vibration signal received from the sensor for detecting vibrations. Then, the signal converter converts it to a digital signal to obtain the first vibration pattern. This pattern is then processed by comparing it to the determined vibration patterns. The output or processed data is communicated via various networks as a command for controlling the notifications device. Particularly, the on-off operation of notifications device, notifying device, target device or communication device with a receiving module, resulting in a notification or connection by transmitting signals to determined application. Examples of notifications may be in many forms, such as sound notification, light notification, text notifications or telephone signal of devices such as a buzzer/ring, computer/tablet, smartphone or other devices with similar working characteristics of relatives, caregivers, medical personnel, etc.

If the first vibration pattern is compatible with the determined vibration patterns, a command will be sent to control the notifications device. For example, if an elderly person falls down within the vibration detection radius of the vibration sensor and the processing unit compares and finds that it is compatible with the determined vibration pattern of falling down, a command will be sent to the notifications device, such as activating a buzzer and/or sending text notifications to the relevant parties specified, etc.

But in case that the first vibration pattern is not compatible with determined vibration patterns, none of command will be sent for controlling notifications device, the result is that none of buzzer works and/or text notification is sent, etc.

### [Notifications Device]

The notifications device is placed on or in contact with a surface in order to detect vibrations from the surface. The vibrated surface can be chosen from the floor or tabletop, as well as floor-like structures or tabletops that can absorb vibrations. The vibration is caused by the force exerted by a part of the body on the surface, such as tapping with the hand, foot, or elbow, stepping or stomping, falling onto the surface, dancing, jumping, etc.

The controlling notifications device can be chosen from sound signaling and light signaling, on and off functionality of device, connecting to designated communication device, device state change, connecting to and transmitting signals to one or many determined applications.

An example of a notifications device is sound signaling to alert a person or caregiver in the vicinity when an abnormal event occurs. The user or elderly person does not need to touch the notifications device but instead relies on the vibration from the surface. For instance, if the elderly person falls down and becomes injured to the point of being unable to move or even losing consciousness, the impact causes the surface to vibrate and triggers the notifications device to emit a sound notification or connect to a communication device such as sending notification messages/emails, or connecting to applications such as LINE for notifications.

In addition, in the event that the notifications device is in an activated state, but there is no actual event or false alarm, the user can rhythmically tap the floor with their hand or foot to turn off or deactivate the device as specified. If the sensor detects a vibration (a second vibration) within a specified period and the processing unit determines that it is compatible with the vibration patterns of the notification deactivation, a command will be sent to the notifications device, such as a buzzer, to deactivate the notification and/or send a message to relevant persons informing them that it was a false alarm, etc.

The systems for controlling notifications device via surface vibrations based on this invention have a high enough resolution to accurately distinguish emergencies such as falling down, (details in Example 1) and can be also used to detect abnormality of footsteps, which may lead to an increase risk of falling down (details in Example 2).

### [Method for Controlling Notifications Device Via Surface Vibrations]

Methods for controlling the notifications device via surface vibrations may have many patterns, including but not limited to, one pattern for controlling the target device by relying on the first vibration pattern from the surface and another pattern for deactivating the notification caused by the first vibration by receiving data of the second vibration pattern from the surface, etc.

In one aspect of this invention, the method for controlling notifications device via surface vibrations comprises:
A. Obtaining data of the first vibration pattern from the surface received from the sensor for detecting vibrations;
B. Processing the first vibration pattern by comparing to determined vibration patterns; and
C. Controlling notifications device that contacts a surface based on the compatibility of the first vibration pattern and determined vibration patterns.

Another aspect of invention on the method for controlling notifications device via surface vibrations may further comprise the transmission of the second vibration pattern to deactivate or change the status of notifications device.

The method for controlling notifications device via surface vibrations comprises receiving the data of the first vibration pattern from the surface obtained from the sensor for detecting vibrations; the processing of the first vibration pattern by comparing with determined vibration patterns; controlling notifications device in contact with the surface. If the first vibration pattern is compatible with the determined vibration patterns and in case that the device has been in the activated state; receiving the data of the second vibration pattern from surface obtained from the sensor for detecting vibrations; the processing of the second vibration pattern by comparing with the determined vibration patterns and controlling notifications device to deactivate the notification. If the second vibration pattern is compatible with determined vibration patterns to deactivate notifications.

The deactivation of notifications is a very useful step because if there is a false alarm during actual use, the user or elderly person can create vibrations or specified vibration patterns on the surface or determined pattern to inform the caregiver or relative that there is no abnormal event or no need for urgent help. The system and methods based on this invention can result in a better quality of life for the elderly and those involved, reducing the likelihood of death or disability without privacy issues like other previous devices or methods. In addition, the usage of the system is not complicated.

The system and methods for controlling the notifications device via surface vibrations based on this invention can be applied to invent notifications device used for the elderly who may fall down or experience any emergency that requires help. The following examples of devices are just application models for understanding and are not limited to the application models in any way. The falling notifications device is just an example of applying the system and methods according to this invention. A person having ordinary skill in the relevant field can modify, improve, reduce, and add any detail or component without going beyond the scope of the annexed claims.

### [Falling Notifications Device]

The falling notifications device comprises an internal structure comprising a geophone sensor or a sensor for detecting vibrations from a surface in contact with the surface, a processing unit which processes the vibration data from the sensor to improve it as a first vibration pattern and compares it to determined vibration patterns, then sends commands for controlling the notifications device and a notifications device with a receiving module that receives commands from the processing unit. The geophone sensor and processing unit are contained inside the structure of the falling notifications device, while the notifications device may be inside or outside the structure.

In one aspect of the falling notifications device with a geophone sensor, processing unit, and a buzzer or sound signalling device inside the structure allows for the mobility of this type of falling notifications device to a room or area where the elderly person lives.

In another aspect of the falling notifications device with a geophone sensor, processing unit, and sound signalling device outside the structure, it may be suitable for serving and taking care of many elderlies where a sound signalling device or buzzer panel can be installed or positioned in a central or designated area (which may be located far away from the elderly). When the receiving module receives commands from the processing unit of the notifications device (in this case, a buzzer), it will echo to notify nurses or caregivers in the central or designated area of any falling, accidents, or emergency situations, resulting in timely assistance for patients or the elderly.

Example of an application of system and methods for controlling notifications device via surface vibrations based on this invention.

### Example 1 Falling Notifications

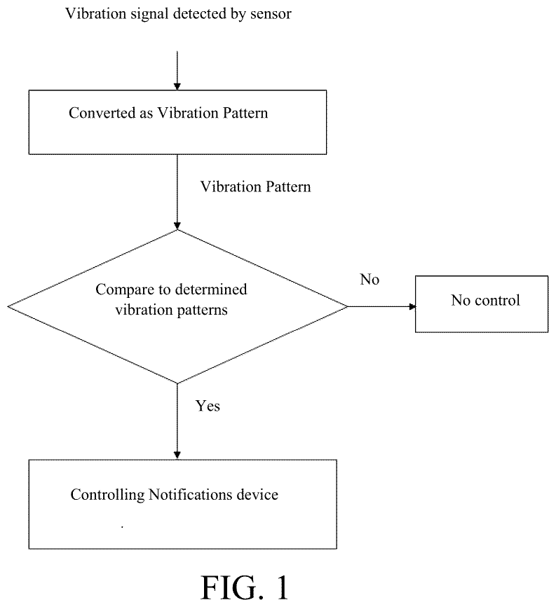

FIG. 1 shows an application pattern with the method of this invention, comprising steps for receiving vibration signals, processing, and comparing vibration patterns to distinguish between the vibrations caused by individual falling and walking at different distances, which obviously have different amplitude, frequency, count, etc. Particularly, the processing unit is designed to compare vibration signals detected by the sensor with a precisely determined vibration signal to determine whether it is characteristic of a falling event or not. If the processing result indicates a falling event, then the notifications device will notify via a telephone signal, sound signal, and/or text message as determined.

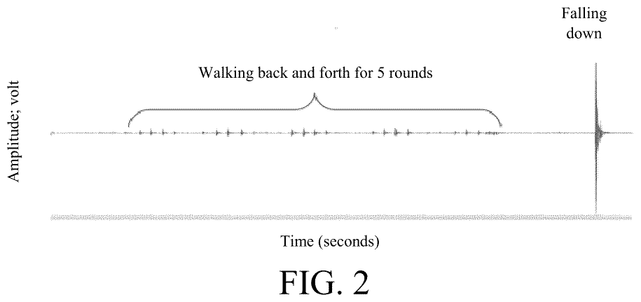

FIG. 2 is a graph showing vibration pattern caused by usual walking back and forth of the subject, which is compared to the vibration pattern caused by the falling.

### Example 2 Notifications for a Change in Walking Style

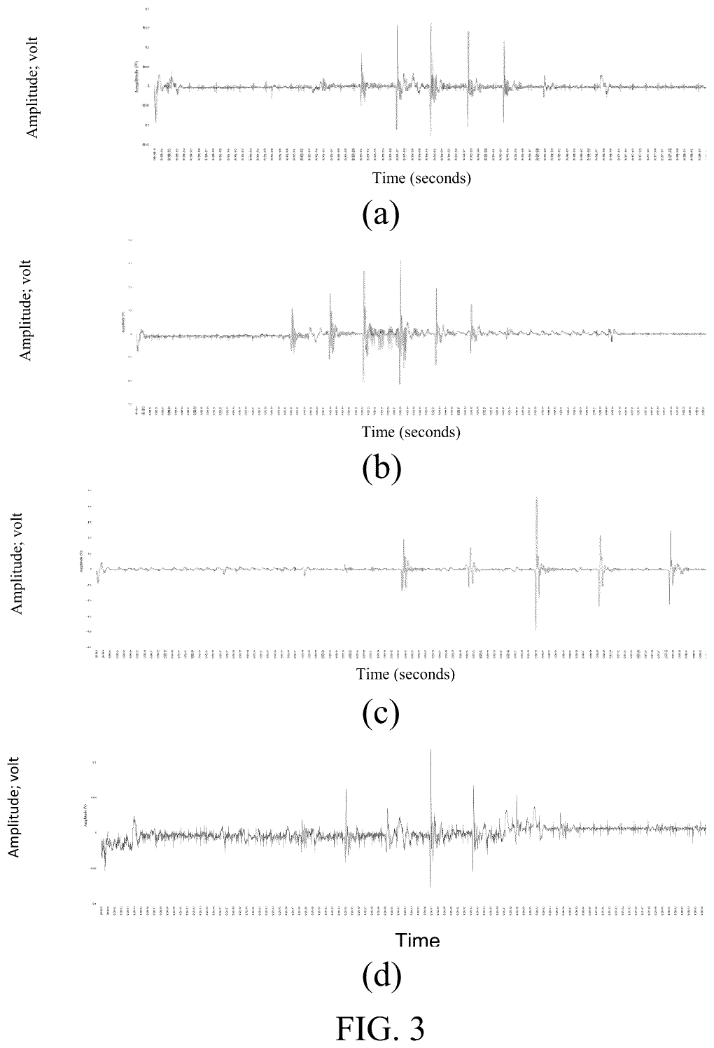

FIG. 3 shows another aspect of the application for the systems according to this invention, which detects and records vibration signals caused by the user's paces. If the pattern changes from the recorded pattern over time in terms of amplitude, frequency, count, etc., the system can notify caregivers of potential dangers caused by diseases related to the joint system and/or gait disorders based on the recorded information and research.

Examples (including but not limited to) include Peripheral sensory ataxia, Hip problems, Spine problems, Arthritis, Joint Problems, Hemiparesis, Paresis, Spasticity from paraplegia, Parkinsonism, Cerebral Ataxia, Ignition failure from frontal lobe, etc., which walking in different ways will cause vibrations, specifically, different vibration patterns.

### Example 3 Actual Use in Residential Area

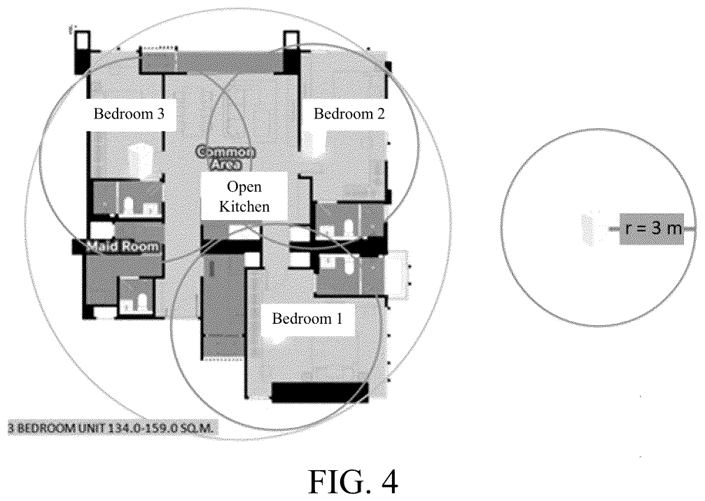

The device can operate in a radius of at least 3 meters or more, depending on the properties of materials used for flooring and building structures, the radius may be up to several tens of meters from the experimental results in certain locations. FIG. 4 shows an example of a vibration detection radius; however, in actual use, room layouts, walls and big-scaled furniture may affect the working distance; therefore, measurements and adjustments are required at the actual site during the installation and the calculation of the number of devices. In practice, areas such as corners or balconies, may not necessarily be covered by the device.

What is claimed is:

1. A system for controlling the notifications device via surface vibrations caused by the subject, comprising:
a sensor for detecting vibrations from a surface in contact with the surface;
a processing unit that processes the vibration data from the sensor to enhance it as a first vibration pattern and compares it to pre-defined vibration patterns, and then sends commands for controlling the notifications device; and
a receiving module that receives commands from the processing unit.

2. The system for controlling the notifications device via surface vibrations caused by the subject according to claim 1, wherein the vibration sensor is geophone sensor.

3. The system for controlling the notifications device via surface vibrations caused by the subject according to claim 1, wherein the vibration surface is from the floor and the top of table.

4. The system for controlling the notifications device via surface vibrations caused by the subject according to claim 1, wherein the surface vibration is caused by the force applied by a part of body of the subject to the surface.

5. The system for controlling the notifications device via surface vibrations caused by the subject according to claim 1, wherein the parameters used to compare vibration patterns comprises frequency, amplitude, count, and interval.

6. The system for controlling the notifications device via surface vibrations caused by the subject according to claim 1, wherein the control of the notifications device is the activation or notifications device deactivation of the notifications device.

7. A method for controlling notifications device via surface vibrations caused by the subject comprises:
receiving the first vibration signal from the surface obtained from the sensor for detecting vibrations;
processing the first vibration signal to the first vibration pattern;
comparing the first vibration pattern to pre-defined vibration patterns;
controlling notifications device based on the compatibility of the first vibration pattern and the pre-defined vibration patterns.

8. The method for controlling notifications device according to claim 7, further comprising the second vibration pattern within specified period to deactivate notifications device.

9. The method for controlling notifications device according to claim 7,
wherein the sensor for detecting vibrations is a geophone sensor.

10. The method for controlling notifications device according to claim 7,
wherein the vibration surface is from the floor and the top of table.

11. The method for controlling notifications device according to claim 7,
wherein the vibration surface is caused by the force applied by a part of body of the subject to the surface.

12. The method for controlling notifications device according to claim 7,
wherein the parameters used to compare vibration patterns comprises frequency, amplitude, count and interval.

13. The method for controlling notifications device according to claim 7,
wherein the control of the notifications device is the activation notifications device or deactivation of the notifications device.

14. The method for controlling notifications device according to claim 7,
wherein the control of the notifications device is any combination of sound signal, light signal, text signal, image signal, and vibration signal.
